(19) United States
(12) Patent Application Publication
(10) Pub. No.: US 2016/0153047 A1
(43) Pub. Date: Jun. 2, 2016

(54) CELL CULTURE ISOLATION AND EXPANSION OF CIRCULATING TUMOR CELLS (CTC) FROM CANCER PATIENTS OR ANIMALS TO DERIVE PROGNOSTIC AND PREDICTIVE INFORMATION AND TO PROVIDE A SUBSTRATE FOR SUBSEQUENT GENETIC, METABOLIC, IMMUNOLOGIC, AND OTHER CELLULAR CHARACTERIZATIONS.

(71) Applicant: Wolfram E. Samlowski, Las Vegas, NV (US)

(72) Inventor: Wolfram E. Samlowski, Las Vegas, NV (US)

(73) Assignee: TrueCells, LLC, Las Vegas, NV (US)

(21) Appl. No.: 14/080,759

(22) Filed: Nov. 14, 2013

(51) Int. Cl. C12Q 1/68; C12N 5/09

(52) U.S. Cl. CPC C12Q 1/6886; C12N 5/0693; C12Q 2600/178; C12Q 2600/156; C12Q 2600/158

(57) ABSTRACT

Cell culture isolation and expansion of CTC from cancer patients to derive prognostic and predictive information and provide a substrate for subsequent genetic, metabolic, immunologic and other cellular characterizations. This method isolates and extracts CTC from a blood sample to grow cancer cell colonies for a long-term tumor repository, tumor biology and pharmacogenomics research. Following removal of small numbers of contaminating host cells, this method will assist with detection of single nucleotide changes in cancer cells to direct treatment, provide global screening for mutations in oncogenes or tumor suppressor genes, and provide a minimally invasive biopsy technique that may allow reassessment of tumors after treatment failures. This method provides an RNA expression screen at a single locus or a genomic level and allow identification of signaling pathways, provide an evaluation of key protein expression to study their metabolic pathways, and provide individual or global screened miRNA molecules.

### CROSS-REFERENCE TO RELATED APPLICATIONS

None

### FEDERALLY SPONSORED RESEARCH

None

### SEQUENCE LISTING

None

### I. BACKGROUND

This relates to cell culture isolation and expansion, specifically cell culture isolation and expansion of circulating tumor cells (CTC) from cancer patients and animals to derive prognostic and predictive information and provide a substrate for subsequent genetic, metabolic, immunologic and other cellular characterizations.

Early during tumor development, cancer cells gain the ability to enter blood vessels, circulate, bind to the lining of blood vessels, extravasate and grow in distant organs.¹ This process is termed “metastasis”. Development of cancer metastases in vital organs is the major cause of cancer-related death.^{2,3} Using sophisticated techniques, rare circulating tumor cells (CTC) can be detected in the blood of patients. These CTC cells are believed to be the source of metastases and eventual patient mortality.

About two decades ago, the presence of CTC in the blood of cancer patients began to be suspected, based on RT-PCR testing of white blood cell samples from cancer patients. These studies employed primers directed against epithelial and melanoma genes (e.g., cytokeratin 19, tyrosinase).⁴ These cancer-derived markers were detectable in the mRNA derived from metastatic cancer patients, but not in blood from normal individuals. Whether this mRNA was derived from intact cells could not be established. These assays lacked specificity and quantitation of PCR product was problematic. Illegitimate expression of genetic markers or pseudogene products derived from normal cells was often detected, confounding the accuracy of the assay.⁴

A number of techniques have been proposed to capture and count CTC. These techniques include: flow cytometry,^{5,6} density gradient based enrichment,⁷ filter-based trapping,^{8,9} immunohistochemical staining of cells on thick smears of buffy coats (enriched peripheral blood white cells),^{10,11} rosette formation, immunomagnetic enrichment,^{12,13} or in microfluidic chambers.¹⁴ Each is technically demanding and difficult to reproduce, since only a tiny number of tumor cells (1-100) can be detected among 10 million white blood cells in 7.5 ml blood.

The only CTC technology currently licensed by the United States FDA, was standardized and commercialized by Veridex for analysis of patient blood samples. The Veridex assay employs physical enrichment of CTC from blood using EpCAM monoclonal antibodies.¹⁵ Blood samples are collected in proprietary “CellSearch® Circulating Tumor Cell (CTC) Kit” blood collection tubes containing preservative and fixative. Samples are then processed on an expensive proprietary cell separation apparatus “CellTracks® AutoPrep® System” using immunomagnetic bead-linked monoclonal antibodies (mAb). Cells are then permeabilized in special chambers and stained with anti-cytokeratin antibodies, prior to semi-automated counting using another expensive piece of equipment “CellTracks Analyzer II®”. Further specificity is achieved by counterstaining leukocytes with CD45 mAb and identifying cell nuclei with DAPI stain.¹⁶ Digital photographs of each potential CTC are prepared for review by the operator of the instrument to verify the accuracy of CTC identification.

Detection and enumeration of cytokeratin-stained, nucleated CTC provides important prognostic information. Using this approach, CTC can be visualized in three types of cancer patients (colon, breast, and prostate), but not in normal controls.¹⁶ Identification of >5 CTC per 7.5 ml blood is believed by Veridex to correlate with a poor prognosis in patients with these cancers.

### Strengths and Weaknesses in the Current FDA Approved CTC Analysis

Using the Veridex EpCAM CTC assay, investigators found that CTC could be detected in patients with regionally advanced or metastatic breast, colon, and prostate cancer.¹⁷⁻¹⁹ The number of CTC correlates strongly with cancer stage, progression and patient survival in breast, colon and prostate cancer.^{17,19,20} Increases in CTC may pre-date radiographic evidence of metastases.^{20,21} Decreases in CTC correlated with eventual decreases in blood PSA and radiologic response in prostate cancer.¹⁸

The EpCAM based CTC assays have serious limitations. Cells are collected into fixative. The fixative kills all living cells. Further immunophenotyping is not possible after fixation. Cell functions cannot be studied after cells are fixed (for example metabolism). When immunomagnetic separation is performed, cells are badly damaged. This results in poor quality mRNA or DNA isolation.

Other problems exist with the EpCAM-based CTC detection. This assay only works for prostate, breast and colon cancer, but not for any other common cancers, like melanoma, and renal cancer, because these cancers don't express EpCAM. Another problem is that 5-15% of breast, colon, and prostate cancers express low levels of EpCAM, making the CTC in this patient group virtually undetectable^{12,22}. Konigsberg et al showed that there is substantial variability in EPCAM expression on the surface of individual CTC, therefore making CTC detection inconsistent or unreliable²³. It is highly likely that a CTC assay specific to these cancers would also correlate with prognosis, and provide a useful marker for treatment response. Therefore, it was attempted to develop a cell-culture based CTC isolation technology, because we thought this would detect all cancer CTCs more effectively.

The CTC detection system has been standardized and commercialized by Veridex, LLC, based on automated sample preparation and semi-automated digital microscopy. Using this assay, investigators found that CTC could be detected in patients with regionally advanced or metastatic breast, colon, and prostate cancer. The number of CTC correlates strongly with cancer stage, progression and patient survival in breast, colon and prostate cancer. Increases in CTC may pre-date radiographic evidence of metastases. CTC assays also have predictive value during treatment. Decreases in CTC correlate with eventual tumor marker and radiologic response in prostate cancer. The FDA approved the use of EpCAM-based CTC assays as a Sprognostic and predictive laboratory test in breast, colon, and prostate cancer. All of these assays share serious limitations: Cells are generally collected into fixative (stable for shipping), which means viable, living cells are not able to be isolated. Cells are badly damaged if immunomagnetic separation is employed, resulting in poor quality mRNA or DNA isolation from the assay. Further immunophenotyping is frequently not possible after fixation. Cell functions cannot be studied after cells are fixed (for example metabolism). In addition, the selection marker EpCAM that is frequently used to isolate CTC may be a cell differentiation marker and may miss the most primitive (stem cell-like) and most lethal circulating tumor cells. This marker may also be variably expressed between different tumors, as well as on cells within the same tumor. EpCAM is only expressed on some epithelial cancers (like breast, colon, prostate and lung) and not many other cancers, such as melanoma and kidney cancer, making it useless in these tumor types.

### Need for a Better CTC Assay

The current commercially available CTC assay (Veridex) is not useful in many types of cancer, such as melanoma, sarcoma, and renal cancer. A significant percentage (5-15%) of individual tumors of patients with metastatic breast, colon, and prostate cancer have cancers that do not express high levels of EpCAM, making tumor cells easy to miss in this assay^{12,22}. It is also suspected that there may be substantial variability in EPCAM expression on individual tumor cells circulating in the same patient²³. Furthermore, there is a strong possibility that the most immature tumor cells that possess “tumor stem cell” properties may not be recognized in this assay at all. CTC are particularly important because they may share these cancer “stem cell” properties and represent the actual tumor cells that are in the act of metastasizing²⁴. The tumor “stem cell” concept remains somewhat controversial^{25,26}, because “stem cells” may not represent a fixed developmental step during hierarchical tumor cell differentiation. They could be dynamically and bidirectionally regulated by the tumor cell microenvironment²⁷. It is highly likely that the least differentiated tumor cells that possess “tumor stem cell” properties are not recognized in EpCAM based CTC assays at all^{28,29}. These very primitive “tumor stem cells” are most likely to cause tumor cell metastases and maintain cancer growth. It is likely that an alternate CTC assay, such as a culture-based assay, would be useful to more accurately establish prognosis, and to provide a more robust intermediate or predictive marker for treatment response.

Further insight into the biology of the important tumor “stem cell” population could also be derived if living, circulating tumor cells could be isolated and expanded substantially in culture. It should be noted, that the assay procedures being developed by TrueCells, LLC are likely to be broadly applicable to cancer types that currently cannot be tested by current assays. Our assay appears to be more sensitive to CTC detection. Thus this assay will be broadly useful, in both humans and most likely it will also be useful (with minor technical adaptations) in most animal species, broadening applicability.

Currently there a relatively few long-term cultured cancer cell lines are used over and over again by lab researchers for drug development and other cancer related studies. Most of these cell lines have been in use for up to 30-50 years. Due to the long length of time they have been removed from their original human host and selected under laboratory culture conditions, it is highly likely that they no longer accurately represent the human cancer they were derived from. In addition, there are many human cancers types for which no cell lines are available at all. By isolating cells from many different patients, without sequential passaging in the lab or in immunocompromised animals, a fresh tumor cell library can be created for drug companies to test new cancer drugs against upon request. TrueCells cultures can be grown at least for a moderate length of time in culture (months) and used in experiments. Additionally, this method can provide the most virulent human cancer cells, which can invade blood vessels, and enter the circulation, unlike the bulk cells in most cancers, thus selecting for cells that are more likely to mediate cancer mortality and are the most important to target with new treatments.

### II. SUMMARY

Based on modifications of murine and human tumor culture techniques originally developed in the lab in 1986, TrueCells, LLC developed an improved and novel method for cell culture isolation and expansion of CTC from the blood of cancer patients and potentially from animals to derive prognostic and predictive information. These cultured CTC can provide a substrate for subsequent genetic, metabolic, immunologic and other cellular characterizations. This will allow a more sensitive and accurate CTC assay for the use of detection and isolation of said tumor cells. This improvement will be broadly useful, in both humans and potentially animal species.

Samples of blood are obtained from patients following signed acknowledgement of informed consent while participating in an IRB approved protocol. TrueCells have identified optimal 10 ml collection tubes. For overnight shipping, acid-citrate-dextrose (ACD) tubes (Cardinal Health) provided the most efficient yield. CPD wax matrix cell preparation tubes (Becton Dickinson) or heparinized “green top” tubes could be substituted.

TrueCells have employed and optimized two strategies to efficiently isolate and identify CTC from 10 ml vacutainer samples of blood. 1) Red blood cells can be lysed directly, using ammonium chloride osmotic lysis. 2) A two-step density separation procedure can be used, transferring the content of each vacutainer (10 ml blood) onto 4 ml of Ficoll/Hypaque (sp gr 1.077-1.080). Followed centrifugation at 2000 rpm for 10 minutes, the buffy coat layer is carefully pipetted off and placed into a 50 ml centrifuge tube and diluted in Hanks balanced salt solution (HBSS) to wash away Ficoll.

Recovered buffy coat cells (white cells+tumor cells) are split into 6-8 replicate cultures per 10 ml tube of blood. These cultures are plated into proprietary TrueCells medium (classified as a Trade Secret, on file, TrueCells, LLC, Las Vegas, Nev.) and cultured in a 5% CO² incubator at 37° C. for 1-4 weeks. The advantage of the TrueCells system is that there is no selection using mAb that might bias the type of cells that are recovered. Likewise, no exogenous cytokines are added to the culture that may drive differentiation or selection of cells.

At the end of the culture period, each culture dish is scored semi-quantitatively (growth or no growth) or quantitatively (tumor colonies/10 ml blood). Tumor cell colonies can then be recovered for further characterization. Colonies can be individually harvested using a sterile micropipette for clonal comparison or an entire plate can be harvested for larger cell yield (e.g., for immunostaining, PCR-based, genomic, or proteomic analyses).

### III. OPERATION OF METHOD

These are some of the tangible results of the TrueCells process and method of cell culture isolation and expansion of CTC from cancer patients. The mere presence of detectable cancer cell colonies in the TrueCells assay is an adverse marker. The data indicates that this correlates directly with the prognosis of the patient. More succinctly, it will correspond directly with the risk of that patient dying of cancer. A more sophisticated analysis would be to count the number of colonies, and their change following treatment. The data strongly suggests that the change number of colonies over the course of treatment is also likely to predict the progression free and overall survival of the patient. For example, an increase in the number of colonies following several rounds of treatment is likely to indicate that the patient's cancer is progressing and that the patient will have shorter survival. Decreases in tumor cell colony formation will indicate that the patient is improving and will have a longer period of response and survival. Thus, this CTC assay is intended to act as an early or intermediate marker for the potential success or failure for a treatment strategy, long before X-rays or lab tests could possibly indicate any changes.

As important as the determination of predictive and prognostic information is in cancer treatment, the usefulness of CTC as a substrate to provide diagnostic and molecular information about a patient's cancer is likely to prove even more powerful. The “Holy Grail” of cancer biology is ability to obtain repeated biopsies of tumor cells from each patient before and after treatment to assess biologic responses to drug treatment. The TrueCells technology has provided a novel tool for obtaining repeated and minimally invasive biopsies of each patient's cancer at various times merely by obtaining samples of peripheral blood. This is a huge advance for cancer scientists and clinicians. Potential applications that are envisioned (not an exhaustive list), include:
a) The isolation and expansion of CTC from each patient suggests the possibility of creating individual vaccines for each patient from proteins derived from their own cancer. In order to function as a vaccine, a patient must present cancer proteins or “antigens” for processing within cells, such as macrophages and dendritic cells via self-MHC (major histocompatibility antigen or HLA antigen) proteins. These proteins are then clipped down to 8-9 amino acid stretches that are then re-expressed back on the surface of macrophages and dendritic cells, carried by the MHC protein. This allows T cells of the immune system to recognize these foreign proteins and potentially react to them. What this means is that different 8-9 amino acid stretches of tumor proteins act as antigens in different individuals, based on the capacity of each individuals HLA proteins (which are usually different) to recognize and bind these peptides. This has meant that at best, a given peptide vaccine could only work in a limited number of individuals of the same HLA type. This has previously meant that hundreds of peptide vaccines would have to be produced for one cancer protein in order to treat everyone with a given cancer effectively. For example, at most 30% of humans share the most common HLA-A antigen, A2. The frequency of other HLA proteins in the population is less frequent. This TrueCells work could provide potential autologous (or self-derived) tumor cells for development of vaccines for many regional disease and most metastatic cancer patients. These cells would have the right tumor antigen, and being derived from the same person, would share the same HLA type. Vaccination with their own tumor (or purified constituents) would allow patients own immune system to select which components to react to, simplifying the preparation process and the need for generation of numerous and complicated reagents, and insuring a high level of successful immunization.
b) Detection of single nucleotide changes in cancer cells to direct treatment. Single nucleotide mutations in cancer causing genes (oncogenes and tumor suppressor genes) are known to both cause cancer to develop, as well as predict behavior of a cancer. Finding or detecting such a mutation in circulating cells may have important implications in establishing a cancer diagnosis. These genetic changes in cancers now represent important targets of new drug development. An important current principal is that patients who have the appropriate mutation will respond to small molecule inhibitor, those that lack will not respond or may even be detrimentally be affected by treatment with the agent.
   i. As an example: 40% of melanoma patients have a specific mutation at position 600 of the B-RAF gene, resulting in a single amino acid change (V600E). These patients will respond dramatically to vemurafenib (Zelboraf) with 70% probability of response, and an improved progression-free and overall survival. There is evidence to suggest treatment of cancer cells lacking this mutation with vemurafenib accelerates their growth! Currently, it can take 4-8 weeks to find tissue blocks and send them for sequencing to find out if patients are eligible to receive this drug. Even more frustrating, sometimes hospitals discard blocks after 7 years or biopsies don't yield adequate diagnostic material, further delaying this important result. TrueCells can isolate enough cells in 4-7 days for PCR-based testing in most patients, improving the rapidity of diagnosis!
c) Cultured CTC can be used for global screening of cancer cells for mutations in oncogenes or tumor suppressor genes (cancer causing genes). Traditionally cancer therapy was assigned based on the appearance of the cancer under the microscope, and similarities to the appearance of cells of normal tissues or organs. However, it has become clear that oncogene mutations may occur across tissue and cancer types. Since there are now hundreds of small molecule inhibitors, it makes sense to attempt large-scale or genome-wide screening of cancer cell DNA for mutations to identify potentially active drugs, regardless of histologic tumor type. For example, identifying “kidney cancer” makes less sense than knowing the patient's cancer may have a rare mutation that activates a specific pathway, such as the MET oncogene, which can be targeted by a number of oral agents. Tumor cell DNA can now be screened for large numbers of mutations at one time by employing mutation-specific microarray chips. Freshly isolated cells from the TrueCells cultures are an ideal starting material for such screens, avoiding many of the potential artifacts of archived paraffin embedded tissue samples.
d) Following treatment of cancers like GI stromal tumors with small molecule inhibitors, additional mutations in key oncogenes, like c-KIT, may eventually occur that allow the patient's tumor to become resistant to the original drug treatment (e.g. imatinib). A minimally invasive, inexpensive biopsy technique can allow reassessment of tumors after treatment failures to identify if one or more new mutations have occurred, and to allow testing of additional inhibitors to see if cells are sensitive (e.g. to a second generation c-KIT inhibitor drug, like nilotinib).
e) Screening RNA expression at a single locus or at a large scale or genomic level may allow identification of which signaling pathways are active in a cancer cell. This may allow a better understanding of how the cancer cell is being regulated to grow and divide, as well as to identify specific drug targeting of that particular pathway, which may be unique to that patient (personalized medicine). This may allow treatment to be tailored to, that individual patient.
f) Cultured CTC can be used for evaluation of key protein expression in cancer cells to study their metabolic pathways. These proteins can be assayed via traditional approaches (such as western blots) or via newer, more generalized proteomic approaches to identify key signaling pathways. For example, most of the ‘oncogenes’ that activate cells into cancer are actually protein kinases that attach a phosphate group to other proteins to activate a signaling cascade in cells. The net result is a change in cell metabolism, growth, and division. These kinases can be assessed phosphoantibody staining, or at a larger scale at the level of the entire “kinome” via a science called metabolomics or proteomics. This may allow assessment of which pathway is driving the growth of a cancer in a given patient.
   Many of the new anticancer agents block a specific kinase, such as the B-RAF (V600E) kinase mentioned previously. Samples of cells provided by TrueCells before and after treatment could demonstrate that a drug is effective in shutting off the signaling pathway following treatment (or that the cells disappear from the circulation because they are being killed). For these experiments, it may be necessary to maintain cultured cells in small amounts of the drug being tested, to maintain inhibition during the culture period. Once a patient progresses or relapses, further testing of the TrueCells product cells could identify which metabolic pathway is taking over from the original signaling pathway driving cancer cell growth to allow escape of the cancer from drug treatment and resulting in drug resistance. This may allow logical identification of additional drug sensitivities and prediction of the best subsequent treatment option from that point forward (another way to personalize cancer therapy).
g) MicroRNA (miRNA) are small pieces of RNA that bind to messenger RNA (mRNA) and prevent the corresponding proteins from being transcribed and produced in cells. Usually each miRNA binds quite a few different mRNA types and thus results in rapid shutdown of cell processes (for example cell cycle). These miRNA molecules turn out to be important in regulating cancer cells, cancer stem cells, cell division, and cell differentiation, and other cell functions. Using cells provided by TrueCells, miRNA molecules can be individually or globally screened (using miRNA chips) before and after treatment, or following other perturbations to study the biology of cancer, or the effects of drug treatment on cancer in vivo.
h) Currently there are a few long-term cultured cancer cell lines, which have been used over-and-over for many years by lab researchers. Most of these cell lines have been in use for 30-50 years. Due to this length of time, it is highly likely that these cancer cell lines no longer accurately represent the human cancer they were derived from. In addition, there are many human cancers types for which no cell lines are available at all. By isolating cells from many different patients, without sequential passaging in the lab or in immunocompromised animals, we can create a fresh tumor cell library for drug companies to test new cancer drugs. TrueCells provides virulent human cancer cells that have already established their ability to invade blood vessels and enter the circulation, unlike the bulk cells in most cancers. A culture technology naturally selects for CTC that are more likely to mediate cancer mortality and are the most important cells to target with new treatments.
i) There are undoubtedly many other applications for viable CTC isolated from individual patients at sequential times during cancer treatment that cannot fully be predicted at this time.

### IV. DESCRIPTION OF FIGURES AND TABLES

FIG. 1 is a flow chart of TrueCells assay for isolating and growing CTC colonies.

FIG. 2 is a tabulation of Melanoma Patient Characteristics.

FIG. 3 is an evaluation of melanoma CTC colonies.

FIG. 4 is a summary of immunostaining of dissociated CTC colonies.

FIG. 5 is a Papanicolaou stain of dissociated melanoma CTC colony cell.

FIG. 6 is a flow cytometry histogram derived from melanoma CTC colony cells and analysis for DNA content.

FIG. 7 is a flow cytometry histogram showing ALDH staining versus DNA content of cultured CTC cells.

FIG. 8 is an identification of tumor cells using forward and side scatter by flow cytometry.

FIG. 9 is an identification of viable breast cancer CTC using labeling by fluorescent C12 lipid staining.

FIG. 10 is the result of CTC cultures performed in a broad spectrum of cancers.

FIG. 11 is an example of single melanoma colonies derived from two different cancer patients (4×).

FIG. 12 is an example of Merkel cell cancer colonies derived from a single patient (4×).

### V. DETAILED DESCRIPTION

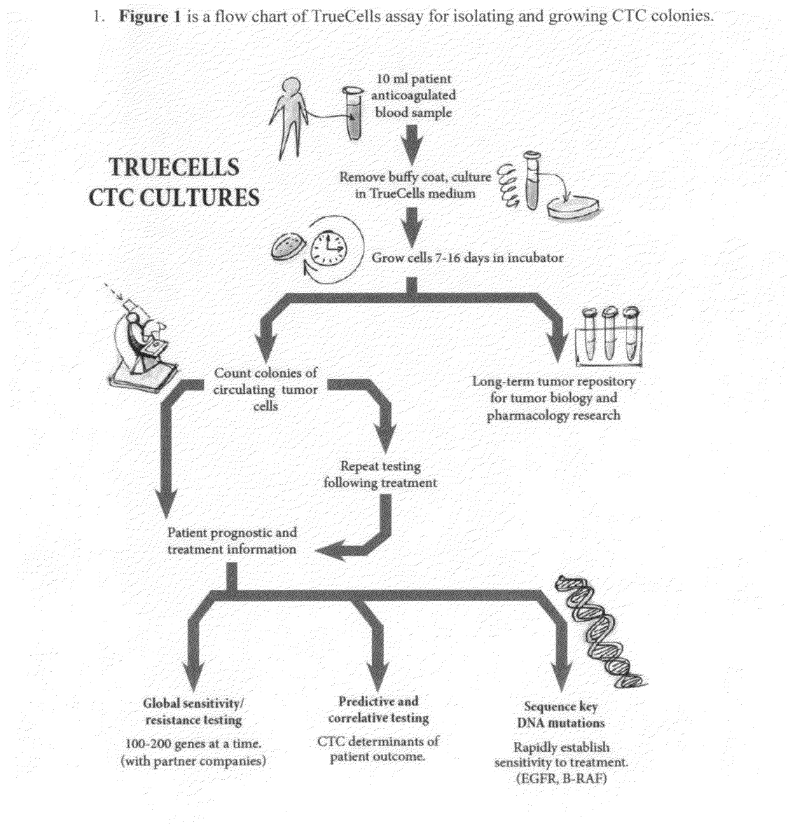

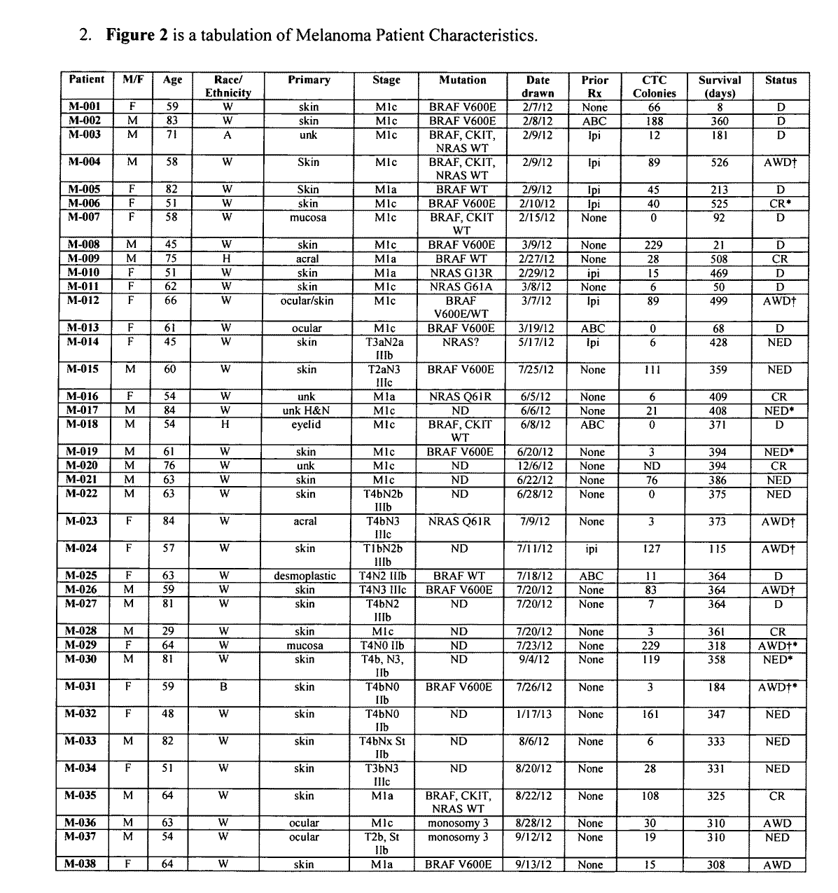

FIG. 1 is a flow chart illustrating a method for isolating, growing and extracting CTC colonies using the TrueCells method and process as outlined. Samples of blood are obtained from patients following signed acknowledgement of informed consent on an IRB approved protocol. Optimal 10 ml blood collection tubes have been identified. For overnight shipping acid-citrate-dextrose (ACD) tubes (Cardinal Health) appeared to provide the most efficient yield, pursuant to current evaluation data, although CPD wax matrix cell preparation tubes (Becton Dickinson) could be substituted. For immediate cell isolation, heparinized “green top” tubes can also be employed.

Two strategies can be used to efficiently isolate the white blood cell fraction for CTC culture from anticoagulated blood collection tubes.
1) Red blood cells can be lysed directly, using ammonium chloride osmotic lysis;
2) A two-step density separation procedure can be used, transferring the content of each vacutainer (7.5 ml blood) onto 4 ml Ficoll/Hypaque (sp gr 1.077-1.080).

This is followed by centrifugation at 2000 rpm for 10 min. The buffy coat layer is carefully pipetted off and cells are extensively washed to remove Ficoll/Hypaque.

All recovered buffy coat cells (white cells+tumor cells) are split into 6-8 replicate cultures per 10 ml tube of blood. These cultures are plated into proprietary TrueCells medium (classified as a Trade Secret, on file at TrueCells, LLC, Las Vegas, Nev.). CTC are then cultured in a 5% CO² incubator at 37° C. for 1-4 weeks. The advantage of this system is that there is no a priori selection with mAb or other processing that might bias the type of tumor cells that are recovered. Likewise, no exogenous cytokines are added to the culture that may drive differentiation or selection of cells.

At the end of the culture period, each culture dish is scored semi-quantitatively (growth or no growth) or quantitatively (the number of tumor colonies per 10 ml blood). Tumor cell colonies can then be recovered for further characterization. Colonies can be individually harvested using a sterile micropipette for clonal comparison or an entire plate can be harvested for larger cell yield (e.g., for immunostaining, PCR-based, genomic, or proteomic analyses).

To validate the identification of actual tumor cells, colonies are isolated using a sterile micropipette and placed onto cytocentrifuge slides. Samples are immunostained with dual immunofluorescent markers to establish lineage identity. Generally, this includes a mAb that represents a specific cancer marker and an antibody directed against white blood cells, each labeled with distinguishing fluorescent dyes to allow separate identification by flow cytometry. Additional markers can also be added, as desired to address research questions.

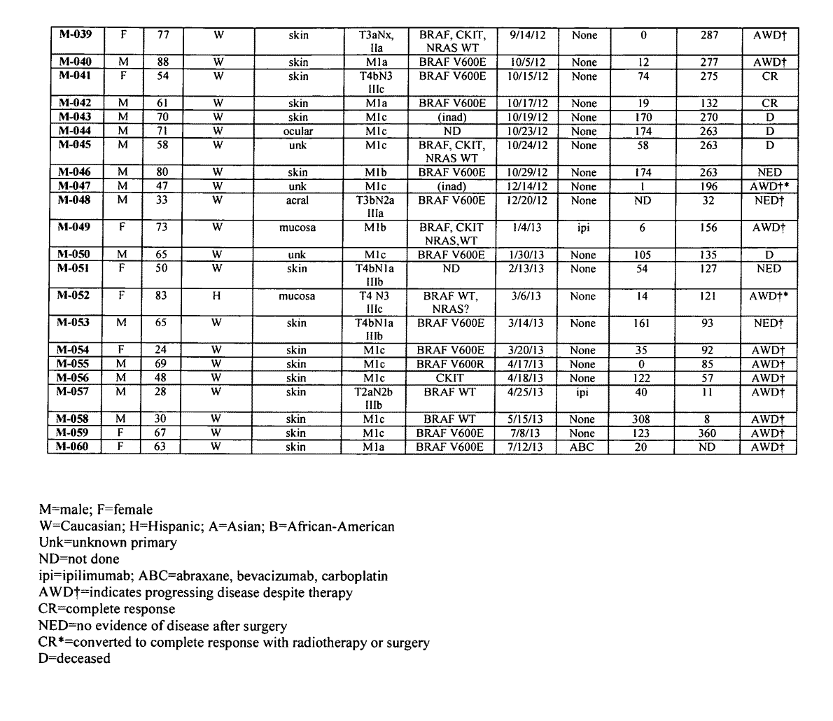

FIG. 2 is reserved. The following table depicts a tabulation of Melanoma
Patient Characteristicsand a Melanoma CTC culture data as proof of concept. .

| | | | Race/ | | | | Date | | Prior | CTC | Survival | |
|---|---|---|---|---|---|---|---|---|---|---|---|---|
| Patient | M/F | Age | Ethnicity | Primary | Stage | Mutation | drawn | | Rx | Colonies | (days) | Status |
| M-001 | F | 59 | W | skin | M1c | BRAF V600E | Feb. 7, | 2012 | None | 66 | 8 | D |
| M-002 | M | 83 | W | skin | M1c | BRAF V600E | Feb. 8, | 2012 | ABC | 188 | 360 | D |
| M-003 | M | 71 | A | unk | M1c | BRAF, CKIT, | Feb. 9, | 2012 | Ipi | 12 | 181 | D |
| | | | | | | NRAS WT | | | | | | |
| M-004 | M | 58 | W | Skin | M1c | BRAF, CKIT, | Feb. 9, | 2012 | Ipi | 89 | 526 | AWD† |
| | | | | | | NRAS WT | | | | | | |
| M-005 | F | 82 | W | Skin | M1a | BRAF WT | Feb. 9, | 2012 | Ipi | 45 | 213 | D |
| M-006 | F | 51 | W | skin | M1c | BRAF V600E | Feb. 10, | 2012 | Ipi | 40 | 525 | CR* |
| M-007 | F | 58 | W | mucosa | M1c | BRAF, CKIT | Feb. 15, | 2012 | None | 0 | 92 | D |
| | | | | | | WT | | | | | | |
| M-008 | M | 45 | W | skin | M1c | BRAF V600E | Mar. 9, | 2012 | None | 229 | 21 | D |
| M-009 | M | 75 | H | acral | M1a | BRAF WT | Feb. 27, | 2012 | None | 28 | 508 | CR |
| M-010 | F | 51 | W | skin | M1a | NRAS G13R | Feb. 29, | 2012 | ipi | 15 | 469 | D |
| M-011 | F | 62 | W | skin | M1c | NRAS G61A | Mar. 8, | 2012 | None | 6 | 50 | D |
| M-012 | F | 66 | W | ocular/skin | M1c | BRAF | Mar. 7, | 2012 | Ipi | 89 | 499 | AWD† |
| | | | | | | V600E/WT | | | | | | |
| M-013 | F | 61 | W | ocular | M1c | BRAF V600E | Mar. 19, | 2012 | ABC | 0 | 68 | D |
| M-014 | F | 45 | W | skin | T3aN2a | NRAS? | May 17, | 2012 | Ipi | 6 | 428 | NED |
| | | | | | IIIb | | | | | | | |
| M-015 | M | 60 | W | skin | T2aN3 | BRAF V600E | Jul. 25, | 2012 | None | 111 | 359 | NED |
| | | | | | IIIc | | | | | | | |
| M-016 | F | 54 | W | unk | M1a | NRAS Q61R | Jun. 5, | 2012 | None | 6 | 409 | CR |
| M-017 | M | 84 | W | unk H&N | M1c | ND | Jun. 6, | 2012 | None | 21 | 408 | NED* |
| M-018 | M | 54 | H | eyelid | M1c | BRAF, CKIT | Jun. 8, | 2012 | ABC | 0 | 371 | D |
| | | | | | | WT | | | | | | |
| M-019 | M | 61 | W | skin | M1c | BRAF V600E | Jun. 20, | 2012 | None | 3 | 394 | NED* |
| M-020 | M | 76 | W | unk | M1c | ND | Dec. 6, | 2012 | None | ND | 394 | CR |
| M-021 | M | 63 | W | skin | M1c | ND | Jun. 22, | 2012 | None | 76 | 386 | NED |
| M-022 | M | 63 | W | skin | T4bN2b | ND | Jun. 28, | 2012 | None | 0 | 375 | NED |
| | | | | | IIIb | | | | | | | |
| M-023 | F | 84 | W | acral | T4bN3 | NRAS Q61R | Jul. 9, | 2012 | None | 3 | 373 | AWD† |
| | | | | | IIIc | | | | | | | |
| M-024 | F | 57 | W | skin | T1bN2b | ND | Jul. 11, | 2012 | ipi | 127 | 115 | AWD† |
| | | | | | IIIb | | | | | | | |
| M-025 | F | 63 | W | desmoplastic | T4N2 IIIb | BRAF WT | Jul. 18, | 2012 | ABC | 11 | 364 | D |
| M-026 | M | 59 | W | skin | T4N3 IIIc | BRAF V600E | Jul. 20, | 2012 | None | 83 | 364 | AWD† |
| M-027 | M | 81 | W | skin | T4bN2 | ND | Jul. 20, | 2012 | None | 7 | 364 | D |
| | | | | | IIIb | | | | | | | |
| M-028 | M | 29 | W | skin | M1c | ND | Jul. 20, | 2012 | None | 3 | 361 | CR |
| M-029 | F | 64 | W | mucosa | T4N0 IIb | ND | Jul. 23, | 2012 | None | 229 | 318 | AWD†* |
| M-030 | M | 81 | W | skin | T4b, N3, | ND | Sep. 4, | 2012 | None | 119 | 358 | NED* |
| | | | | | IIb | | | | | | | |
| M-031 | F | 59 | B | skin | T4bN0 | BRAF V600E | Jul. 26, | 2012 | None | 3 | 184 | AWD†* |
| | | | | | IIb | | | | | | | |
| M-032 | F | 48 | W | skin | T4bN0 | ND | Jan. 17, | 2013 | None | 161 | 347 | NED |
| | | | | | IIb | | | | | | | |
| M-033 | M | 82 | W | skin | T4bNx St | ND | Aug. 6, | 2012 | None | 6 | 333 | NED |
| | | | | | IIb | | | | | | | |
| M-034 | F | 51 | W | skin | T3bN3 | ND | Aug. 20, | 2012 | None | 28 | 331 | NED |
| | | | | | IIIc | | | | | | | |
| M-035 | M | 64 | W | skin | M1a | BRAF, CKIT, | Aug. 22, | 2012 | None | 108 | 325 | CR |
| | | | | | | NRAS WT | | | | | | |
| M-036 | M | 63 | W | ocular | M1c | monosomy 3 | Aug. 28, | 2012 | None | 30 | 310 | AWD |
| M-037 | M | 54 | W | ocular | T2b, St | monosomy 3 | Sep. 12, | 2012 | None | 19 | 310 | NED |
| | | | | | IIb | | | | | | | |
| M-038 | F | 64 | W | skin | M1a | BRAF V600E | Sep. 13, | 2012 | None | 15 | 308 | AWD |
| M-039 | F | 77 | W | skin | T3aNx, | BRAF, CKIT, | Sep. 14, | 2012 | None | 0 | 287 | AWD† |
| | | | | | IIa | NRAS WT | | | | | | |
| M-040 | M | 88 | W | skin | M1a | BRAF V600E | Oct. 5, | 2012 | None | 12 | 277 | AWD† |
| M-041 | F | 54 | W | skin | T4bN3 | BRAF V600E | Oct. 15, | 2012 | None | 74 | 275 | CR |
| | | | | | IIIc | | | | | | | |
| M-042 | M | 61 | W | skin | M1a | BRAF V600E | Oct. 17, | 2012 | None | 19 | 132 | CR |
| M-043 | M | 70 | W | skin | M1c | (inad) | Oct. 19, | 2012 | None | 170 | 270 | D |
| M-044 | M | 71 | W | ocular | M1c | ND | Oct. 23, | 2012 | None | 174 | 263 | D |
| M-045 | M | 58 | W | unk | M1c | BRAF, CKIT, | Oct. 24, | 2012 | None | 58 | 263 | D |
| | | | | | | NRAS WT | | | | | | |
| M-046 | M | 80 | W | skin | M1b | BRAF V600E | Oct. 29, | 2012 | None | 174 | 263 | NED |
| M-047 | M | 47 | W | unk | M1c | (inad) | Dec. 14, | 2012 | None | 1 | 196 | AWD†* |
| M-048 | M | 33 | W | acral | T3bN2a | BRAF V600E | Dec. 20, | 2012 | None | ND | 32 | NED† |
| | | | | | IIIa | | | | | | | |
| M-049 | F | 73 | W | mucosa | M1b | BRAF, CKIT | Jan. 4, | 2013 | ipi | 6 | 156 | AWD† |
| | | | | | | NRAS,WT | | | | | | |
| M-050 | M | 65 | W | unk | M1c | BRAF V600E | Jan. 30, | 2013 | None | 105 | 135 | D |
| M-051 | F | 50 | W | skin | T4bN1a | ND | Feb. 13, | 2013 | None | 54 | 127 | NED |
| | | | | | IIIb | | | | | | | |
| M-052 | F | 83 | H | mucosa | T4 N3 | BRAF WT, | Mar. 6, | 2013 | None | 14 | 121 | AWD†* |
| | | | | | IIIc | NRAS? | | | | | | |
| M-053 | M | 65 | W | skin | T4bN1a | BRAF V600E | Mar. 14, | 2013 | None | 161 | 93 | NED† |
| | | | | | IIIb | | | | | | | |
| M-054 | F | 24 | W | skin | M1c | BRAF V600E | Mar. 20, | 2013 | None | 35 | 92 | AWD† |
| M-055 | M | 69 | W | skin | M1c | BRAF V600R | Apr. 17, | 2013 | None | 0 | 85 | AWD† |
| M-056 | M | 48 | W | skin | M1c | CKIT | Apr. 17, | 2013 | None | 122 | 57 | AWD† |
| M-057 | M | 28 | W | skin | T2aN2b | BRAF WT | Apr. 25, | 2013 | ipi | 40 | 11 | AWD† |
| | | | | | IIIb | | | | | | | |
| M-058 | M | 30 | W | skin | M1c | BRAF WT | May 15, | 2013 | None | 308 | 8 | AWD† |
| M-059 | F | 67 | W | skin | M1c | BRAF V600E | Jul. 8, | 2013 | None | 123 | 360 | AWD† |
| M-060 | F | 63 | W | skin | M1a | BRAF V600E | Jul. 12, | 2013 | ABC | 20 | ND | AWD† |

M = male;
F = female
W = Caucasian;
H = Hispanic;
A = Asian;
B = African-American
Unk = unknown primary
ND = not done
ipi = ipilimumab;
ABC = abraxane, bevacizumab, carboplatin
AWD† = indicates progressing disease despite therapy
CR = complete response
NED = no evidence of disease after surgery
CR* = converted to complete response with radiotherapy or surgery
D = deceased

We have tested this novel and efficient technique for culture of CTC from blood samples from 58 melanoma patients. Ninety percent of the high-risk (stage IIb-IV) melanoma patients grew colonies containing tumor cells from their blood. The number of colonies isolated per patient ranged from 0-308, with a mean of 63±9.5 (SEM). The individual patient characteristics of the confirmatory patient cohort are shown. Sixty melanoma patients signed consent to provide samples, although two patients never actually had samples drawn. The actual study cohort was 32 men and 26 women (58 patients). There were 53 Caucasians, 3 Hispanics, 1 Asian and 1 African-American. There were 43 skin melanomas, 5 unknown primary melanomas, 4 ocular, 4 mucosal, and 2 acral melanomas. There were 7 Stage II patients (1 stage IIa, 6 stage IIb), 14 Stage III (Stage IIIb-8, Stage IIIc-6) and 37 Stage IV (Stage IVa-9, Stage IVb-2, Stage IVc-26).

Of the study cohort, 22 had BRAF mutations (21 V600E, 1 V600R). There were 6 patients with NRAS mutations and 1 with a C-KIT mutation. Biopsies failed to provide adequate tissue for sequencing in 3 patients and 12 patients were wild type at all loci tested. Tumors from 14 Stage II and III patients were not sent for sequencing. Fifty-two of fifty-eight high-risk melanoma patients grew colonies from their blood samples (90%). The patients who did not grow CTC colonies represented patients who were in remission (2 patients) or had very recently received anticancer therapy within a week of the blood draw (2 patients), potentially interfering with colony outgrowth. Only two samples failed to grow colonies in patients with significant tumor burden for unclear reasons, which could represent possible technical failures. Ten normal volunteers (people without cancer) also provided blood samples under a IRB protocol and had a mean colony growth of 0.5±1.4 colonies. The rare colonies seen in normal volunteers appeared to be mature adipose cells that result from using vacutainer tubes to draw blood.

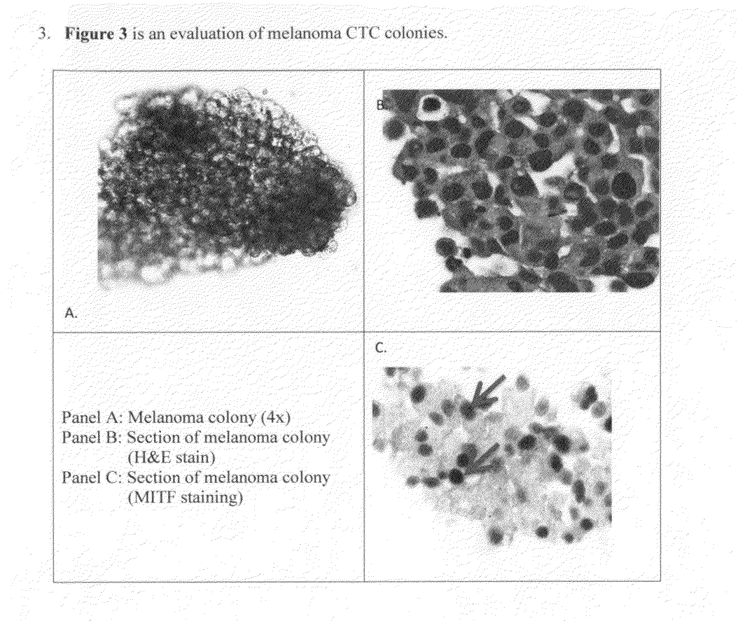

FIG. 3 depicts an evaluation of melanoma CTC colonies. Seven normal volunteers had virtually no colony growth (0.5±1.4 colonies). Intact colonies were harvested with a micropipette. Tumor colonies were embedded in paraffin, sectioned, and stained with melanoma-specific mAb.

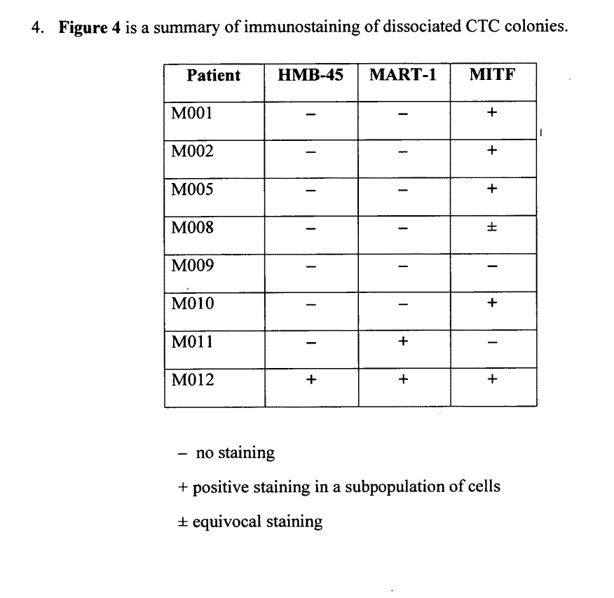

FIG. 4 depicts a summary of immunostaining of dissociated CTC colonies in a three-panel relief. Panel A depicts a Melanoma colony at approximately 4× magnification. Panel B depicts a section of melanoma colony (H&E stain). Panel C depicts a section of melanoma colony (MITF staining). The most consistent melanoma mAb immunostain proved to be MITF, a melanoma stem cell marker. Tumor colonies were harvested, washed in isotonic saline and briefly incubated with Accutase to produce a single cell suspension. After additional washes, 50 μl of the cell suspension was allowed to settle onto a glass slide for 30 minutes. After media was gently decanted and blotted, slides were air-dried and fixed in 95% ethanol. Cells were stained with the monoclonal antibody shown using a BenchMark Ultra Staining system and the Optiview DAB hapten detection system.

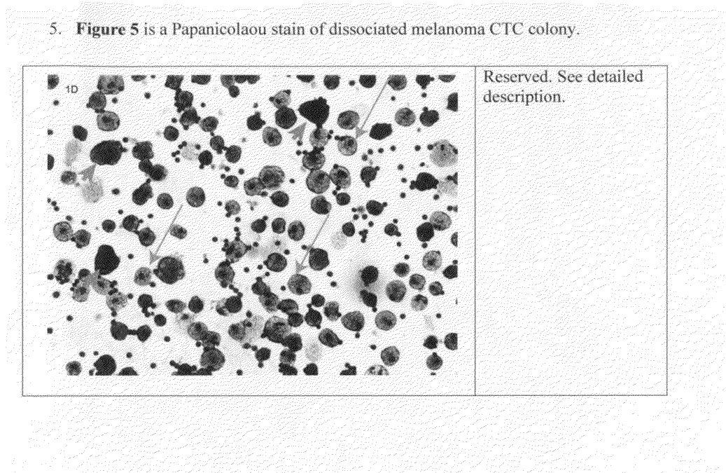

FIG. 5 depicts a Papanicolaou stain of dissociated melanoma CTC colony. Papanicolaou stain of dissociated melanoma CTC colony showing tumor cells (green arrows) and host macrophages (orange arrows) on a background of smaller host cells. A host cell component in CTC colonies was also observed. This raised an issue of how host and tumor cells within the colonies could specifically be identified, quantified, and then separated. Following enzymatic dissociation of colonies in Accutase, Papanicolau staining readily distinguished host (green arrows) and tumor cells (orange arrows), based on the abnormal nuclei of tumor cells in the CTC colonies.

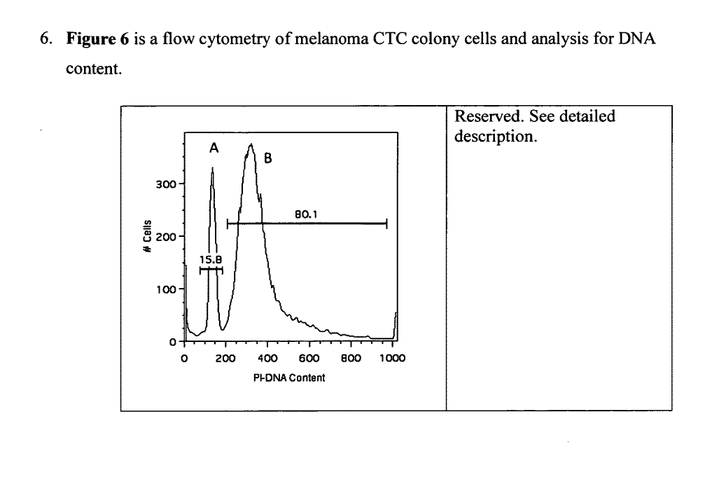

FIG. 6 depicts a flow cytometry of melanoma CTC colony cells and analysis for DNA content and characterization of tumor cells within TrueCells CTC colonies. Flow cytometry demonstrated cells within colonies that were diploid (normal DNA content). Colonies also contained aneuploid cells, which have markedly increased DNA content (a characteristic of cancer cells). Fixed and permeabilized cells (BD Cytofix/Cytoperm) were treated with 200 μg/ml RNAse (R4875, Sigma, St. Louis, Mo.) for 10 minutes. Propidium iodide (PI, P4864, Sigma St. Louis, Mo.) was added at a final concentration of 50 μg/ml. Twenty thousand events were analyzed per sample using a FACSCalibur flow cytometer and data was analyzed using FlowJo (Ashland, Oreg.) software. Data was plotted showing PI fluorescence on a linear scale. Normal human leukocytes (diploid DNA) or M14 melanoma cells (aneuploid DNA) served as controls. DNA histogram derived from dissociated colonies grown from patient M-058. These were stained with propidium iodide, showing a quantitatively smaller host cell diploid DNA peak (A) and a larger aneuploid tumor cell peak (B).

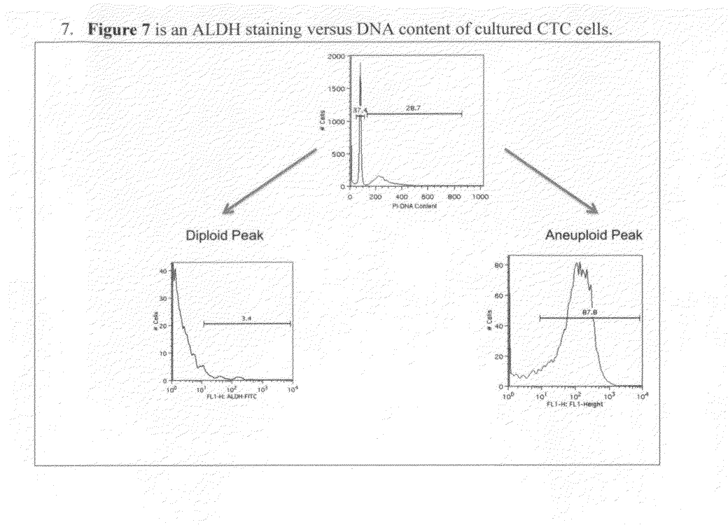

FIG. 7 depicts an ALDH staining versus DNA content of cultured CTC cells. Only the aneuploid cells expressed the stem cell marker ALDH1A1. This marker was not present on diploid cells. Cells were stained for either ALDHIA1 (sc-374076, Santa Cruz Biotechnology, Dallas, Tex.) using a FITC labeled anti-mouse secondary antibody for detection (sc-2010 Santa Cruz) at 5 μg/ml for 30 minutes, followed by extensive washes. Cells were subsequently fixed and permeabilized, then stained with PI as described above. The cells were analyzed using a FACSCalibur flow cytometer (BD Biosciences, San Jose, Calif.) at the UNLV Genomics Core facility. Data analysis was subsequently performed using Flow-Jo software (TreeStar, Inc.). Unstained cells and cells stained with secondary antibody alone served as negative controls for flow cytometric gating.

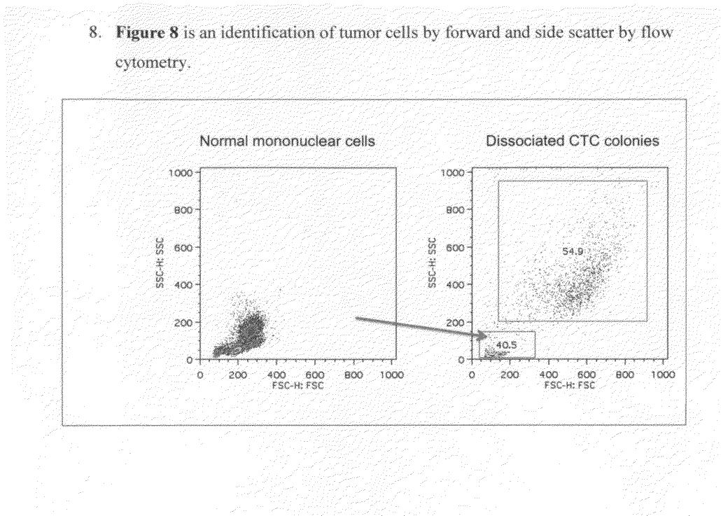

FIG. 8 depicts an identification of tumor cells by forward and side scatter by flow cytometry. After Accutase digestion, cell smears were prepared by allowing 50 μl of a single cell suspension to settle onto a glass slide. After media was gently decanted and blotted, slides were air-dried and fixed in 95% ethanol. Twenty thousand events were analyzed for forward and side scatter per sample using a FACSCalibur flow cytometer and data was analyzed using FlowJo (Ashland, Oreg.) software. The tumor cells were larger and more granular than host mononuclear cells (upper square). These properties allow clear distinction between host and tumor cells. Normal human leukocytes or M14 melanoma cells served as controls to set gating.

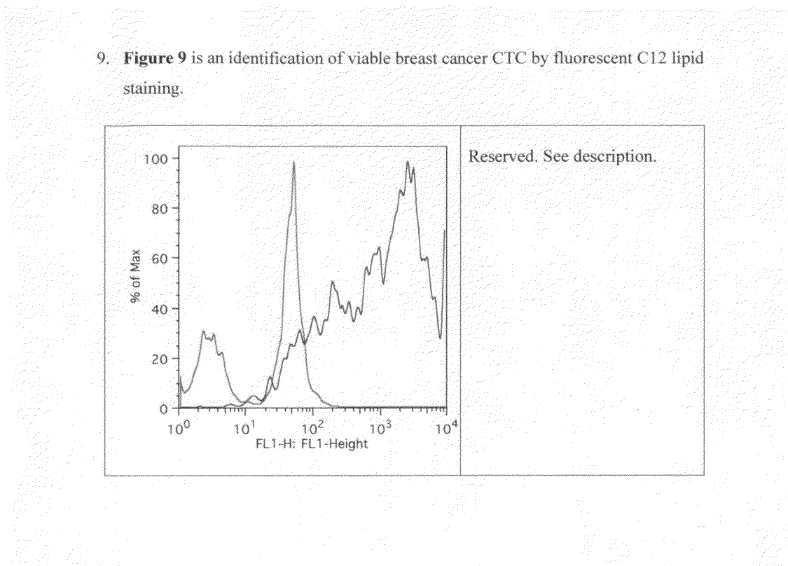

FIG. 9 depicts an identification of viable breast cancer CTC by fluorescent C12 lipid staining. Samples of dissociated CTC colony cells from breast cancer B-007 stained with C12-BODIPY (blue) versus control (red). This panel shows the striking increase in fluorescence related to the uptake of the lipid probe. This staining procedure allows clear identification of viable cancer cells from melanoma, breast cancer and other tumors based on their increase in fluorescence. This staining procedure will allow separation of host and tumor cells by fluorescence-activated cell sorting, if further enrichment of tumor cells is required. CTC colony cells were washed once Hanks Balanced Salt Solution (HBSS, Sigma, St. Louis, Mo.) and incubated for 10 minutes in 10 ng/ml of 4,4-Difluoro-5-Methyl-4-Bora-3a, 4a-Diaza-s-Indacene-3-Dodecanoic Acid (C12-BODIPY 500/510; D3823; Life Sciences) at 37° C. Cells were washed once with PBS and permeabilized using BD Cytofix/Cytoperm, as described for PI co-staining. Samples were subsequently analyzed by flow cytometry for both cytoplasmic fluorescence and DNA analysis to establish that only the aneuploid cells stained with lipid.

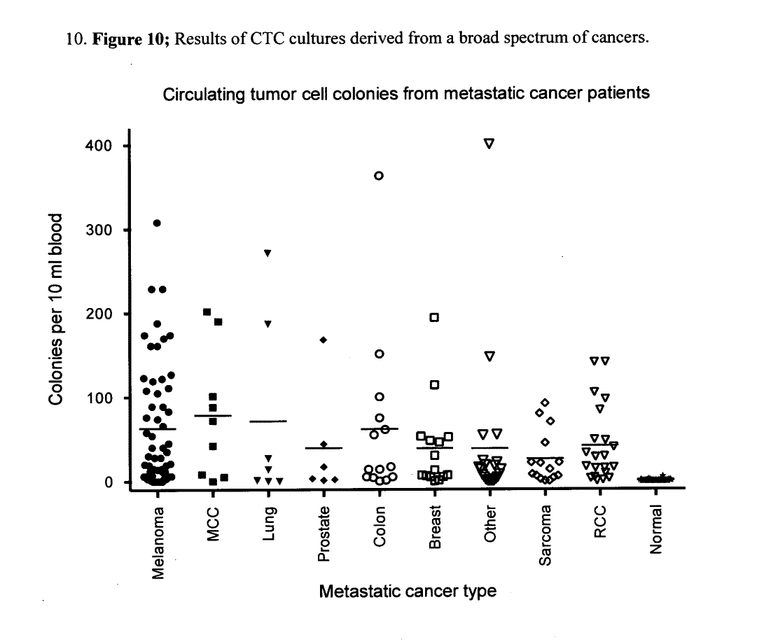

FIG. 10 depicts results of CTC cultures from a broad spectrum of cancers. CTC cultures from melanoma patients were examined under a dissecting microscope on day 16 of culture and colonies were counted. We have subsequently tested this approach in a broad variety of other tumor types, including prostate cancer, sarcoma, renal cancer, Merkel Cell skin cancer and many others. Over 90% of patients with metastatic cancer grew tumor cell colonies in the TrueCells Assay. The number of colonies and the mean is shown for each group.

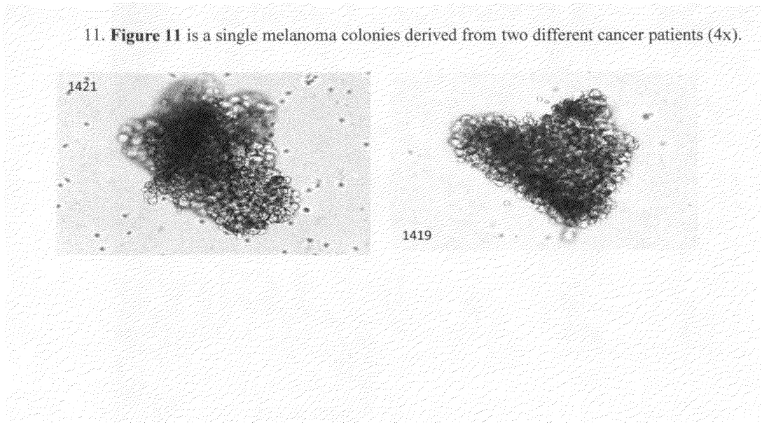

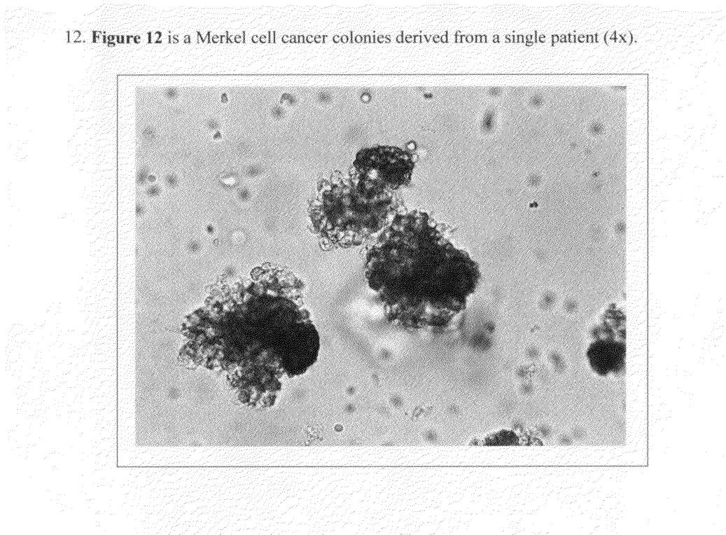

FIG. 11 depicts a single melanoma colonies derived from two different cancer patients at approximately 4× magnification. Photographs of sample tumor cell colonies isolated from the blood of cancer patients are shown in FIGS. 11 and 12 respectively.

FIG. 12 depicts a Merkel cell cancer colonies derived from a single patient at approximately 4× magnification. Photographs of sample tumor cell colonies isolated from the blood of cancer patients are shown in FIGS. 11 and 12 respectively.

### IX. REFERENCES

1. Bernards R, Weinberg R A: A progression puzzle. Nature 418:823, 2002
2. Jemal A, Siegel R, Ward E, et al: Cancer statistics, 2009. CA Cancer J Clin 59:225-49, 2009
3. Pantel K, Alix-Panabieres C: The clinical significance of circulating tumor cells. Nat Clin Pract Oncol 4:62-3, 2007
4. Alunni-Fabbroni M, Sandri M T: Circulating tumour cells in clinical practice: Methods of detection and possible characterization. Methods 50:289-97, 2010
5. Takao M, Takeda K: Enumeration, characterization, and collection of intact circulating tumor cells by cross contamination-free flow cytometry. Cytometry. Part A: the journal of the International Society for Analytical Cytology 79:107-17, 2011
6. Galanzha E I, Shashkov E V, Spring P M, et al: In vivo, noninvasive, label-free detection and eradication of circulating metastatic melanoma cells using two-color photoacoustic flow cytometry with a diode laser. Cancer research 69:7926-34, 2009
7. He W, Kularatne S A, Kalli K R, et al: Quantitation of circulating tumor cells in blood samples from ovarian and prostate cancer patients using tumor-specific fluorescent ligands. International journal of cancer. Journal international du cancer 123:1968-73, 2008
8. Lin H K, Zheng S, Williams A J, et al: Portable filter-based microdevice for detection and characterization of circulating tumor cells. Clinical cancer research: an official journal of the American Association for Cancer Research 16:5011-8, 2010
9. Clawson G A, Kimchi E, Patrick S D, et al: Circulating tumor cells in melanoma patients. PLoS One 7:e41052, 2012
10. Ross A A, Cooper B W, Lazarus H M, et al: Detection and viability of tumor cells in peripheral blood stem cell collections from breast cancer patients using immunocytochemical and clonogenic assay techniques. Blood. 82:2605-2610, 1993
11. Simpson S J, Vachula M, Kennedy M J, et al: Detection of tumor cells in the bone marrow, peripheral blood, and apheresis products of breast cancer patients using flow cytometry. Exp. Hematol. 23:1062-1068, 1995
12. Rao C G, Chianese D, Doyle G V, et al: Expression of epithelial cell adhesion molecule in carcinoma cells present in blood and primary and metastatic tumors. International journal of oncology 27:49-57, 2005
13. Cristofanilli M, Hayes D F, Budd G T, et al: Circulating tumor cells: a novel prognostic factor for newly diagnosed metastatic breast cancer. J Clin Oncol 23:1420-30, 2005
14. Ozkumur E, Shah A M, Ciciliano J C, et al: Inertial focusing for tumor antigen-dependent and -independent sorting of rare circulating tumor cells. Sci Transl Med 5:179ra47, 2013
15. Balzar M, Winter M J, de Boer C J, et al: The biology of the 17-1A antigen (Ep-CAM). J Mol Med 77:699-712, 1999
16. Allard W J, Matera J, Miller M C, et al: Tumor cells circulate in the peripheral blood of all major carcinomas but not in healthy subjects or patients with nonmalignant diseases. Clin Cancer Res 10:6897-904, 2004
17. Cristofanilli M, Budd G T, Ellis M J, et al: Circulating tumor cells, disease progression, and survival in metastatic breast cancer. N Engl J Med 351:781-91, 2004
18. de Bono J S, Scher H I, Montgomery R B, et al: Circulating tumor cells predict survival benefit from treatment in metastatic castration-resistant prostate cancer. Clin Cancer Res 14:6302-9, 2008
19. Cohen S J, Punt C J, Iannotti N, et al: Relationship of circulating tumor cells to tumor response, progression-free survival, and overall survival in patients with metastatic colorectal cancer. J Clin Oncol 26:3213-21, 2008
20. Goodman O B, Jr., Fink L M, Symanowski J T, et al: Circulating tumor cells in patients with castration-resistant prostate cancer baseline values and correlation with prognostic factors. Cancer Epidemiol Biomarkers Prev 18:1904-13, 2009
21. Allen-Mersh T G, McCullough T K, Patel H, et al: Role of circulating tumour cells in predicting recurrence after excision of primary colorectal carcinoma. Br J Surg 94:96-105, 2007
22. Sieuwerts A M, Kraan J, Bolt J, et al: Anti-epithelial cell adhesion molecule antibodies and the detection of circulating normal-like breast tumor cells. J Natl Cancer Inst 101:61-6, 2009
23. Konigsberg R, Obermayr E, Bises G, et al: Detection of EpCAM positive and negative circulating tumor cells in metastatic breast cancer patients. Acta Oncol 50:700-10, 2011
24. Zhang L, Ridgway L D, Wetzel M D, et al: The identification and characterization of breast cancer CTCs competent for brain metastasis. Sci Transl Med 5:180ra48, 2013
25. Quintana E, Shackleton M, Foster H R, et al: Phenotypic heterogeneity among tumorigenic melanoma cells from patients that is reversible and not hierarchically organized. Cancer Cell 18:510-23, 2010
26. Santini R, Vinci M C, Pandolfi S, et al: HEDGEHOG-GLI Signaling Drives Self-Renewal and Tumorigenicity of Human Melanoma-Initiating Cells. Stem Cells 30:1808-18, 2012
27. Ni C, Huang J: Dynamic regulation of cancer stem cells and clinical challenges. Clin Transl Oncol 15:253-8, 2013
28. Scheel C, Weinberg R A: Phenotypic plasticity and epithelial-mesenchymal transitions in cancer and normal stem cells? Int J Cancer 129:2310-4, 2011
29. Joosse S A, Pantel K: Biologic challenges in the detection of circulating tumor cells. Cancer Res 73:8-11, 2013

We claim:

1. A procedure and methodology comprising of:
a. A method to obtain repeated fluid biopsies of tumor cells from the blood of each patient before and after treatment to assess the number of colonies grown in culture. This serves as a prognostic marker and a predictive indicator of a patient's biologic response to treatment;
b. A method of CTC isolation and extraction and culture from patient blood samples;
c. Global screening for mutations in oncogenes or tumor suppressor genes;
d. A minimally invasive biopsy technique that may allow reassessment of tumors after treatment failures;
e. RNA or DNA expression screen at a single locus or at a large scale or genomic level that may allow identification of signaling pathways that are active in a cancer cell;
f. Evaluation of key protein expression in cancer cells to study their metabolic pathways;
g. Individually or globally screened miRNA molecules, using miRNA chips, before and after treatment; and
h. An option to create individual vaccines for each patient from proteins derived from their own cancer.
i. Creation of new cancer cell lines or a tumor cell repository as a basis for further biological discovery.

2. A method of claim 1 further comprising of:
a. CTC assay is intended to act as an early or intermediate marker for the potential success or failure for a treatment strategy;
b. Provide potential autologous (or self-derived) tumor cells for development of vaccines for many individual cancer patients;
c. Using and utilizing CTC colonies to detect single nucleotide mutations in cancer causing genes (oncogenes and tumor suppressor genes);
d. Using and utilizing cultured CTC for global screening of cancer cells for mutations in oncogenes or tumor suppressor genes (cancer causing genes);
e. Allow reassessment of tumors after treatment failures to identify if one or more new mutations have occurred;
f. Allow testing of additional inhibitors to determine if cells are sensitive to the prescribed treatment;
g. Demonstrate that a drug is effective in shutting off the signaling pathway following prescribed treatment; and
h. Using and utilizing cultured CTC for evaluation of key protein expression in cancer cells to study their metabolic pathways.

3. A method of claim 1 and claim 2 further comprising of:
a. Isolation and evaluation of molecules that can be individually or globally screened (using miRNA chips) before and after treatment;
b. Following other perturbations to study the biology of cancer, or the effects of drug treatment on cancer in vivo;
c. Providing virulent human cancer cells that have already established their ability to invade blood vessels and enter the circulation; and
d. Providing culture technology that naturally selects for CTC tumor cells that are more likely to mediate cancer mortality and are the most important cells to target with new treatment development.
e. Provide techniques to assess animal cancers in a manner parallel to human tumors.
